(19) United States
(12) Patent Application Publication
(10) Pub. No.: US 2015/0010431 A1
(43) Pub. Date: Jan. 8, 2015

(54) Method of killing insects and their larvae and spores using a combination of high temperature steam and liquid nitrogen

(71) Applicant: Douglas Burke, Newport Beach, CA (US)

(72) Inventor: Douglas Burke, Newport Beach, CA (US)

(21) Appl. No.: 13/987,159

(22) Filed: Jul. 5, 2013

(51) Int. Cl. A61L 2/22

(52) U.S. Cl. CPC A61L 2/22
USPC 422/26; 422/292

(57) ABSTRACT

A method and system of killing objects wherein said objects are selected from the group consisting of pests, insects, fungus and spores wherein said objects have a starting temperature, T_{O}, and said method and system comprising a first step being application of a means of raising the temperature of said objects by the exposure to heat and water vapor until said objects have reached a temperature T_{H} above said starting temperature, and said method and system comprising a second step being application of a means of lowering the temperature of said objects until said objects have reached a temperature T_{C} below the starting temperature, and said objects being rendered lifeless after experiencing the change in temperatures with respect to time.

### BACKGROUND OF THE INVENTION AND PRIOR ART

Many scientists have used cryogenics in particular liquid nitrogen to kill insects or bugs. U.S. Pat. No. 4,160,336 to Query uses a combination of insecticide and refrigerant. U.S. Pat. No. 4,597,217 to Narita uses liquid nitrogen to kill the field mouse by way of oxygen depletion. U.S. Pat. No. 5,165,199 to Tallon uses liquid nitrogen for exterminating drywood pests such as termites and weevils.

For the purpose spores these treatments are ineffective because the spores can easily withstand being frozen without being killed. This being because they are largely dehydrated.

Liquid nitrogen by itself can be a very expensive way of treating wood because the specific heat of the wood is large and it can take a lot of nitrogen to reduce the temperature of the wood.

The proposed invention overcomes these difficulties and limitations.

### OBJECTS AND ADVANTAGES

(1) The proposed invention can kill seeds and spores whereas many spores and seeds can survive simple freezing.
(2) The proposed invention involves a first application of hot water steam which is capable of diffusing into small cracks in wood to get to insects or spores that reside below the surface of the wood. Thus when the refrigerant is applied the item (pest or spore) will have heat removed from it more quickly than the wood because the thermal conductivity of water is greater than the wood.
(3) When used on logs the proposed invention because of the initial treatment of hot water steam allows for the refrigerant to suck heat out of the pests and spores without having to freeze the entire log or a large fraction of the log, this being because the thermal conductivity of water is greater than the wood.
(4) The proposed invention allows for the bloating and immobilization of living insects from the initial steam treatment so that that when the refrigerant is applied the pest has a harder time running away from the applied refrigerant.
(5) The proposed invention does not employ the use of chemicals that are harmful to humans by way of chemical toxicity.

### BRIEF DESCRIPTION OF THE DRAWINGS

Each of the drawings is a sketch of a micrograph taken of portions of treated and untreated seeds. The sketches are presented in place of the micrographs because the micrographs are blurry and are not electronically reproducible.

FIG. 1: A sketch of a portion of a seed that has not been treated.

FIG. 2: A sketch of a portion of a seed treated only with liquid nitrogen.

FIG. 3: A sketch a portion of a seed treated with hot water vapor steam and then liquid nitrogen.(This combination being the method of the proposed invention)

FIG. 4: A sketch of the top of a seed that has been with the method of the proposed invention wherein which a hole has been formed in the seed.

### DESCRIPTION OF THE INVENTION

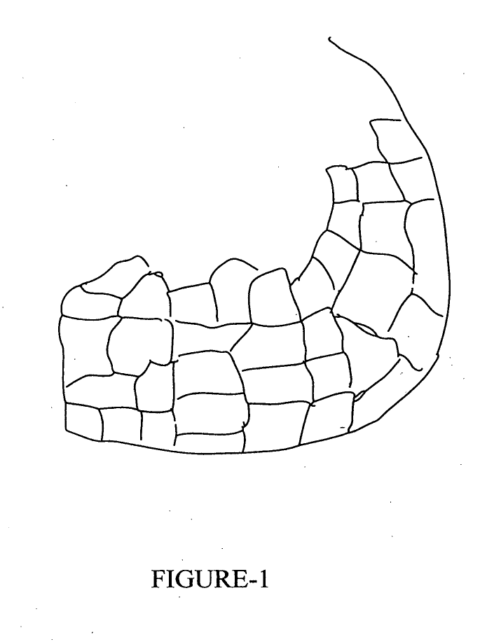

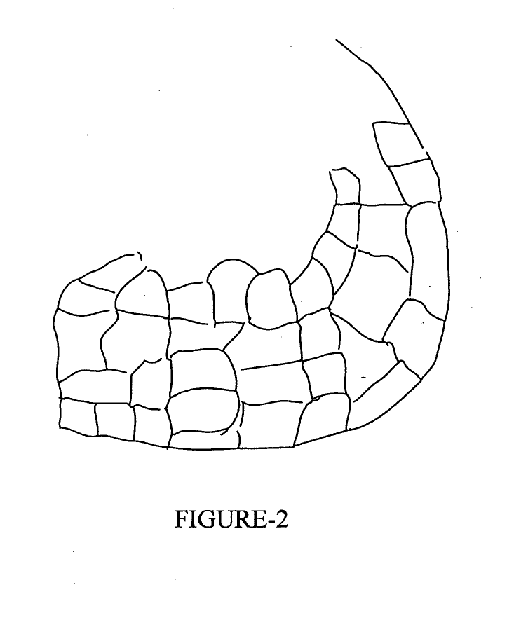

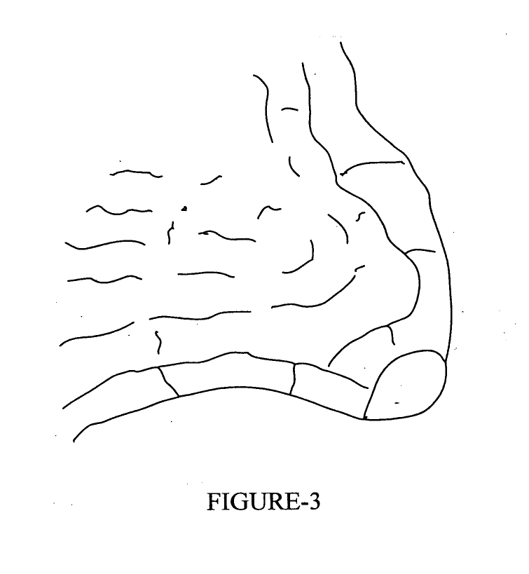

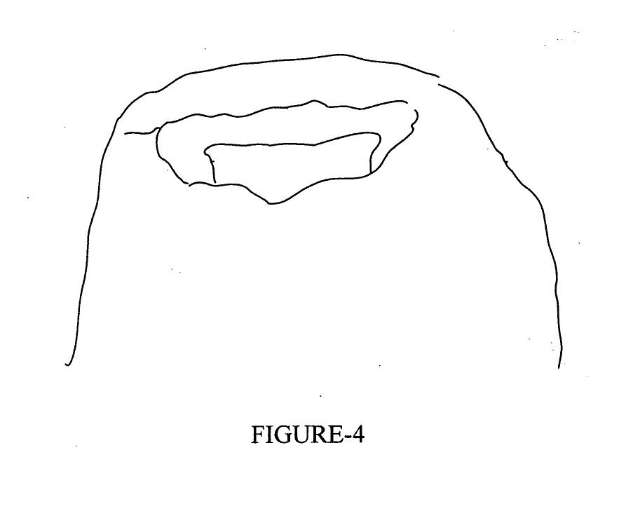

The proposed invention overcomes the limitations of these earlier techniques by incorporating a pre-treatment of high temperature steam (water vapor) followed up by an immediate treatment of a refrigerant such as liquid nitrogen. The hot steam causes the insect or spore to absorb water by way of thermal diffusion. After the insect or spore is bloated with water the refrigerant is applied. Upon lowering the temperature to at least the freezing point of water the thermal expansion of the water causes damage to the biological structure and upon thawing the insect or spore is dead. FIG. 1 shows a sketch of a robust thick walled seed with no treatment. FIG. 2 is a sketch of a seed treated with liquid nitrogen alone. As can be seen there is no real visible damage to the seed. FIG. 3 shows a sketch of a seed treated with hot water vapor steam (temperature greater than or equal to 212 degrees Fahrenheit) and then liquid nitrogen. As can be seen there is damage to the cell walls. The boundaries between cell walls for many of the cells have been destroyed. Also there is distortion to the size and shape of the cells and correspondingly to the entire seed itself. FIG. 4 shows a sketch of a close-up of a hole in the seed caused by the treatment of the proposed invention.

In applications for killing spores, when exposed to the hot water vapor the spore absorbs water and begins its life cycle. It would be unobvious to apply hot steam to a spore to kill it since exposing it to a warm moist environment is what starts its life cycle. So anyone wanting to kill it would not induce life. Thus however when followed with the immediate freezing the earlier exposure to the hot steam augments the effects of freezing. So the method of the proposed invention is to first raise the temperature of the pests or spores with hot water vapor and get them to absorb water. Then the temperature of the pests or spores is lowered and as the bloated pests or spores are frozen the expansion of the water breaks them apart. There are some pests such as cockroaches that can withstand freezing without dying. The proposed invention provides a means of killing even the most robust of pests and spores.

The invention is particularly useful in treating wood in the form of logs because the application of hot steam serves a secondary purpose other than just hydrating the pests and spores. That secondary purpose is that the water has a higher thermal conductivity than the wood. So when the refrigerant is applied to the wood the heat will flow out of the water bloated insect, pest, or spore much more quickly than it flows out of the wood. Therefore the freezing of the pest or spore is accomplished without having to freeze the entire log. This is useful because having to freeze the entire log is costly and makes cryogenics by itself economically ineffective for exterminating pests and spores on logs or whole shipping containers full of logs.

The method can be optimized specific to treating shipping containers full of logs wherein the first treatment comprises the hot (100 degrees Celsius or hotter) water vapor steam being sprayed from below with high pressure exposing the surface of the logs to the hot steam. The second treatment is the application of the refrigerant which is sprayed from above. This allows for the out channels of the refrigerant to not be clogged with water and thereby be frozen shut etc.

Thus the invention comprises a method of killing pests and spores comprising exposing the pests and spores to heat and moisture thereby raising their temperature above the ambient temperature in which they exist and applying moisture so they absorb water. The means of raising the temperature can be by the application of hot water vapor steam. The temperature of the steam needs to be high enough so that the pests and spores absorb water and become bloated. The spores and pests need to be exposed to the hot steam (water vapor) long enough so that they absorb a critical amount of water. The pests and spores then have their temperature lowered to zero degrees Celsius or lower. This lowering of temperature can be achieved by exposing them to a refrigerant. The refrigerant can be liquid nitrogen. When at the freezing temperatures of water the water in the pest or spore expands and fractures the biological tissue. The critical amount of water that needs to be absorbed is that amount such that when frozen the pest or spore is rendered lifeless. In general the spore will absorb an amount of water such that the volume of the spore increases by twenty percent the critical amount will have been achieved so that when frozen lethal biological fracture to the tissues will occur.

In one of the modes of operation of the invention the hot water vapor steam and the refrigerant can be applied one after the other causing the temperature of the objects of interest (pests and spores) to oscillate. This induced oscillation in temperature causes thermal heat to flow in and out of the objects. This thermal shock also can cause enough damage to kill the objects.

The objects in question (pests and spores) have characteristic surface areas A, specific heats C, and thermal conductivities, K. The characteristic time required to raise or lower their temperature from a starting temperature T_{O} to a final temperature T_{F} wherein the thermal reservoir they are in contact with is either the steam of a refrigerant (liquid nitrogen) can be found by the following analysis. The thermal reservoir temperature can be denoted as T_{R}. The size of the object can be approximated as a sphere of radius r. If the thermal reservoir is liquid nitrogen and one is trying to cool the object the temperature of the object is approximately governed by the equation:

$$CM \frac{d T}{d t} = - \frac{KA}{r} ( {T}_{R} - T )$$

If the thermal reservoir is hot water steam and one is trying to heat the object the temperature of the object is approximately governed by the equation:

$$CM \frac{d T}{d t} = \frac{KA}{r} ( {T}_{R} - T )$$

The cooling time of the object to go from a starting temperature T_{O} to a cold temperature T_{C} is given by:

$$\frac{CMr}{KA} ln \frac{( {T}_{O} - {T}_{R} )}{( {T}_{C} - {T}_{R} )}$$

The heating time of the object to go from a starting temperature T_{O} to a hot temperature T_{H} is given by:

$$\frac{CMr}{KA} ln \frac{( {T}_{O} - {T}_{R} )}{( {T}_{R} - {T}_{H} )}$$

If the heating agent is hot water vapor steam then T_{R} is equal to 100 deg Celsius. If the cooling agent is liquid nitrogen then T_{R} is equal to −204 deg Celsius or 77 deg Kelvin.

These expressions are solutions to the relevant differential equation and are approximations. Nevertheless the idea of a heating time and a cooling time is important.

In one of the modes of the invention the objects are heated with steam and cooled with a refrigerant. The heating takes the objects (pests and spores) to a high temperature T_{H} above the starting ambient temperature and the cooling takes the objects (pests and spores). The objects are heated and cooled a plurality of times. This provides repetitive thermal shock and enhances the damage to the biologic tissue of the pests and spores.

In yet another mode of the invention the objects are exposed to a flow of hot water vapor steam from one direction and cold liquid nitrogen from an opposing direction. Thus the objects experience a dramatic flow of heat through them which can cause mechanical fracture to the biological tissue cells and thereby render them lifeless.

While particular embodiments have been shown and described, it is apparent, that changes and modifications may be made without departing from the broader scope, and, therefore, the aim in the appended claims is to cover all such changes and modifications as falls in the true spirit of the present invention.

What is claimed is:

1. A method and system of killing objects wherein said objects are selected from the group consisting of pests, insects, fungus and spores wherein said objects have a starting temperature, T_{O}, and said method and system comprising a first step being application of a means of raising the temperature of said objects by the exposure to heat and water vapor until said objects have reached a temperature TH above said starting temperature, and said method and system comprising a second step being application of a means of lowering the temperature of said objects until said objects have reached a temperature T_{C} below the starting temperature, and said objects being rendered lifeless after experiencing the change in temperatures with respect to time.

2. The method and system of claim one wherein the rate of change of the objects' temperature with respect to time is high enough to render said objects lifeless.

3. The method and system of claim one wherein said first step comprises the application of hot water vapor steam to said objects and said hot water vapor steam has a temperature of at least 100 degrees Celsius

4. The method and system of claim one wherein said first step comprises the application of hot water vapor steam to said objects and said hot water vapor steam has a temperature of at least 100 degrees Celsius, and said application of hot water vapor steam is carried out for a time long enough for said objects to absorb an amount water so that their volume increases.

5. The method and system of claim one wherein said first step comprises the application of hot water vapor steam to said objects and said hot water vapor steam has a temperature of at least 100 degrees Celsius, and said application of hot water vapor steam is carried out for a time long enough for said objects to absorb an amount water so that their volume increases and T_{C} is a temperature that is equal to or less than the freezing point of the water, zero degrees Celsius.

6. The method and system of claim one wherein said first step comprises the application of hot water vapor steam to said objects and said hot water vapor steam has a temperature of at least 100 degrees Celsius, and said application of hot water vapor steam is carried out for a time long enough for said objects to absorb an amount water so that their volume increases by at least ten percent and T_{C} is a temperature that is equal to or less than the freezing point of the water, zero degrees Celsius.

7. The method and system of claim one wherein said first step comprises the application of hot water vapor steam to said objects and said hot water vapor steam has a temperature of at least 100 degrees Celsius, and said application of hot water vapor steam is carried out for a time long enough for said objects to absorb an amount of water, and T_{C} is a temperature that is equal to or less than the freezing point of the water, zero degrees Celsius, and said amount of water is absorbed is an effective amount to to render said objects lifeless after said second step is carried out and said absorbed water is frozen, and said objects are rendered lifeless by way of the expansion of water upon freezing, said thermal expansion from the inside out of said objects causing damage to the cellular structure of said objects.

8. The method and system of claim one wherein said first step comprises the application of hot water vapor steam to said objects and said hot water vapor steam has a temperature of at least 100 degrees Celsius, and said second step comprises the application of liquid nitrogen, and T_{C} is a temperature that is equal to or less than the freezing point of the water, zero degrees Celsius.

9. The method and system of claim one wherein said objects are occupying the surface and subsurface of carrier objects and, said first step comprises the application of hot water vapor steam to said objects and said hot water vapor steam has a temperature of at least 100 degrees Celsius, and the application of said hot water vapor steam provides a thermal link to said objects because the thermal conductivity of said water is higher than the thermal conductivity of carrier, and thus in carrying out steps to raise and lower the temperature of said objects the temperatures of said objects can be raised and lowered by and amount δT, and the temperature of said carrier object is raised by and amount less than δT, and thus the cost of raising and lowering the temperatures of said objects when attached to said carrier object is reduced because of said first step.

10. The method and system of claim one wherein said objects are occupying the surface and subsurface of carrier objects and, said first step comprises the application of hot water vapor steam to said objects and said hot water vapor steam has a temperature of at least 100 degrees Celsius, and the application of said hot water vapor steam provides a thermal link to said objects because the thermal conductivity of said water is higher than the thermal conductivity of carrier, and thus in carrying out steps to raise and lower the temperature of said objects the temperatures of said objects can be raised and lowered by and amount δT, and the temperature of said carrier object is raised by and amount less than δT, and thus the cost of raising and lowering the temperatures of said objects when attached to said carrier object is reduced because of said first step, and said carrier object is selected from the group consisting of lumber, logs, and plant material.

11. The method and system of claim one wherein said first step comprises the application of hot water vapor steam to said objects and said hot water vapor steam has a temperature of at least one hundred degrees Celsius, and said second step comprises the application of liquid nitrogen, and and T_{H} is a temperature that is equal to or greater than one hundred degrees celsius, and T_{C} is a temperature that is equal to or less than the freezing point of the water, zero degrees Celsius.

12. The method and system of claim one wherein said first step comprises the application of hot water vapor steam to said objects and said hot water vapor steam has a temperature of at least one hundred degrees Celsius, and said second step comprises the application of liquid nitrogen, and and T_{H} is a temperature that is equal to or greater than one hundred degrees celsius, and T_{C} is a temperature that is equal to seventy seven degrees Kelvin.

13. The method and system of claim one wherein said first step comprises the application of water vapor, and said second step comprises the application of a refrigerant, and and T_{H} is a temperature that is greater than T_{O}, and T_{C} is a temperature that is equal to or less than the freezing point of the water, zero degrees Celsius, and said water vapor further includes nutrients dissolved therein, and said nutrients are selected from the group consisting of those molecules that promote the absorption of water by spores.

14. The method and system of claim one wherein said first step comprises the application of water vapor, and said second step comprises the application of a refrigerant, and and T_{H} is a temperature that is greater than T_{O}, and T_{C} is a temperature that is equal to or less than the freezing point of the water, zero degrees Celsius, and said first step is carried out for a period of time long enough for said spores to absorb water and begin their life cycle.

15. The method and system of claim one wherein said first step comprises the application of water vapor, and said second step comprises the application of a refrigerant, and and T_{H} is a temperature that is greater than T_{O}, and T_{C} is a temperature that is equal to or less than the freezing point of the water, zero degrees Celsius, and said water vapor further includes sugars dissolved therein, and said sugars are selected from the group consisting of those molecules that promote the intake of water by pests and insects thereby enhancing the intake of water by said insects and pests and thereby increasing their volume.

16. The method and system of claim one wherein said first step comprises the application of hot water vapor steam to said objects and said hot water vapor steam has a temperature of at least 100 degrees Celsius, and said second step comprises the application of liquid nitrogen, and T_{C} is a temperature that is equal to or less than the freezing point of the water, zero degrees Celsius, and said objects have an average cooling time being the time required to reduce their temperature to T_{C} by way of said second step, and said objects have an average heating time being the time required to increase their temperature to T_{H} by way of said first step, and said first step is carried out for a time longer than said heating time, and said second step is carried out for a time longer than said cooling time, and said first and second steps are repeated a plurality of times.

17. The method and system of claim one wherein said first step comprises the application of hot water vapor steam to said objects and said hot water vapor steam has a temperature of at least 100 degrees Celsius, and said second step comprises the application of liquid nitrogen, and T_{C} is a temperature that is equal to or less than the freezing point of the water, zero degrees Celsius, and said objects have an average cooling time being the time required to reduce their temperature to T_{C} by way of said second step, and said objects have an average heating time being the time required to increase their temperature to T_{H} by way of said first step, and said first step is carried out for a time longer than said heating time, and said second step is carried out for a time longer than said cooling time, and said first and second steps are repeated a plurality of times, and said plurality of times comprises an amount effective as required to render the majority of said objects lifeless.

18. A method and system of killing objects wherein said objects are selected from the group consisting of pests, insects, fungus and spores wherein said objects have a starting temperature, T_{O}, and said method and system comprising the application of hot water vapor steam to said objects and said hot water vapor steam has a temperature of at least 100 degrees Celsius, and said application of hot water vapor steam being from a first oriented direction towards said objects, and said steam being applied under pressure so that said steam flows towards said objects in a given first direction, and said method and system further including the simultaneous application of a refrigerant to said objects and said refrigerant has a temperature of at least less than T_{O}, and said application of refrigerant being from a second oriented direction towards said objects, and said refrigerant being applied under pressure so that said refrigerant flows towards said objects in a given second direction, thereby said objects being exposed to a thermal gradient causing heat flow there through said objects and said heat flow causing thermo-mechanical fracture to the structure of said objects thereby rendering a portion of said objects to be lifeless.

19. The method and system of claim eighteen wherein said refrigerant is liquid nitrogen.

20. The method and system of claim eighteen wherein said heated water vapor steam is replaced with a heated dry air stream.

21. The method and system of claim eighteen wherein said heated water vapor steam is replaced with a heated dry air stream, and said refrigerant is liquid nitrogen.

22. The method and system of claim one wherein said first step comprises the application of hot water vapor steam to said objects and said hot water vapor steam has a temperature of at least 100 degrees Celsius and further includes a surfactant wherein said surfactant is selected from the group consisting of water soluble surfactants.
